(19) United States
(12) Patent Application Publication
(10) Pub. No.: US 2025/0170307 A1
(43) Pub. Date: May 29, 2025

(54) LOW FRICTION AND FLEXIBLE CATHETER LINER

(71) Applicant: TELEFLEX LIFE SCIENCES LLC, Wilmington, DE (US)

(72) Inventors: Philip O'MALLEY, Co Clare Cullenagh (IE); Patrick CAMPION, Co. Clare Mountshannon (IE); David BUTLER, Co. Limerick Ballyneety (IE); Sean LYNN, Co Limerick Limerick (IE); Mihir GORADIA, Co Limerick Limerick (IE); Nathaniel Lee Randy RHODES, Maple Grove, MN (US); Greg GABAY, New Hope, MN (US)

(21) Appl. No.: 18/715,641

(22) PCT Filed: Dec. 2, 2022
(86) PCT No.: PCT/IB2022/061728

§ 371 Date: May 31, 2024

Related U.S. Application Data
(60) Provisional application No. 63/285,428, filed on Dec. 2, 2021.

(51) Int. Cl. A61L 29/04; A61L 29/02; A61L 29/14; A61L 29/18; A61M 25/00; A61M 25/01

(52) U.S. Cl. CPC A61L 29/041; A61L 29/02; A61L 29/14; A61L 29/18; A61M 25/0045; A61M 25/0053; A61M 25/0108; A61L 2400/10; A61M 2025/0046

(57) ABSTRACT

A catheter and method for producing a catheter having an inner layer having a polyolefin- or polyethylene-based thermoplastic elastomeric material; an outer layer; and a tie layer disposed between the inner layer and the outer layer. The tie layer is a low durometer polymer. The thermoplastic elastomeric material includes at least one of a polyolefin-based thermoplastic elastomer, including, in one embodiment, a polyethylene-based thermoplastic elastomer. The tie layer includes a maleic anhydride grafted linear low-density polyethylene.

### CROSS REFERENCE TO RELATED APPLICATION

This application claims the benefit of U.S. Provisional Application No. 63/285,428, filed Dec. 2, 2021, the disclosure of which is incorporated herein by reference in its entirety.

### TECHNICAL FIELD

The present invention generally relates to catheters with low friction and flexible liners.

### BACKGROUND

Existing PTFE catheter liners have low friction properties that can allow for delivery of implants or devices and can allow low friction passage of coaxial inner catheters. Nevertheless, many existing PTFE catheter liners are too stiff for some applications and there exists a need for more flexible catheter liners that maintain or improve upon the low friction properties of existing PTFE liners.

### SUMMARY

These needs are met, to a great extent, by the disclosed catheter comprising an inner layer comprising a thermoplastic elastomeric material; an outer layer; and a tie layer disposed between the inner layer and the outer layer. The tie layer comprises a low durometer polymer.

In a first embodiment of the invention, a catheter comprises an inner layer comprising a thermoplastic elastomeric material; an outer layer; and a tie layer disposed between the inner layer and the outer layer, wherein the tie layer comprises a low durometer polymer. The thermoplastic elastomeric material comprises at least one polyolefin-based thermoplastic elastomer. The thermoplastic elastomeric material may comprise at least one polyolefin-based thermoplastic elastomer. The tie layer may comprise a maleic anhydride grafted linear low-density polyethylene. A reinforcement may be disposed between the inner layer and the outer layer. The reinforcement may be embedded in the tie layer. The reinforcement may terminate a fixed distance from a distal end point of the catheter. The outer layer may comprise a first section and a second section, wherein a stiffness of the first section is different from a stiffness of the second section. The tie layer may comprise a first tie layer and a second tie layer. In an further embodiment, the inner layer may comprise a first inner layer on a distal portion of the catheter, the first inner layer comprising the thermoplastic elastomeric material, and a second inner layer disposed on a proximal portion of the catheter proximal to the first inner layer, wherein the second inner layer comprises PTFE. In an embodiment, the inner layer is disposed only on a distal portion of the catheter. In another embodiment, the inner layer is disposed on a full length of the catheter.

In an embodiment of the invention, a catheter comprises an inner layer comprising a polyolefin- or polyethylene-based thermoplastic elastomeric material; an outer layer; and a tie layer disposed between the inner layer and the outer layer, wherein the tie layer comprises a low durometer polymer. The inner layer can comprise one or more lubricants. The tie layer can comprise a maleic anhydride grafted linear low-density polyethylene (LLDPE). The tie layer can comprise a maleic anhydride modified low density polyethylene (LDPE). The tie layer can comprise a maleic anhydride modified ethylene vinyl acetate (EVA).

The catheter can further comprise a reinforcement disposed between the inner layer and the outer layer. The reinforcement can include a single coil wire wrapped circumferential about a longitudinal axis of the catheter. The reinforcement can include multiple filar coil wires wrapped circumferentially about a longitudinal axis of the catheter. The reinforcement can be a braided design wire pattern interwoven circumferentially around a longitudinal axis of the catheter. The braided design can include changes in density or pix per inch along a length of the braided design. The braided design can include a constant density along a length of the braided design. The braided design can include a pattern of 1 under 1 over 1, 2 under 2 over 2, or 1 under 2 over, or other various interweaving strand patterns. The braided design can include 8, 16, or 32 individual strands, or any number of individual strands. The reinforcement can include a geometry that is round, rectangular, or elliptical. The reinforcement can comprise at least one of steel, nitinol, tungsten, non-metallic monofilaments, fibrous bundles, Aramid, polymers, Nylon, or LCP. The reinforcement can include a circumferential reinforcement between the inner layer and the outer layer and a longitudinal reinforcement that extends along a longitudinal axis of the catheter. The longitudinal reinforcement can comprise at least one of steel, nitinol, tungsten, non-metallic monofilaments, fibrous bundles, Aramid, polymers, Nylon, or LCP. The longitudinal reinforcement can include one, two, three, four, or more longitudinal reinforcements, or any number. The longitudinal reinforcement can include a plurality of longitudinal reinforcements arranged symmetrically around a central longitudinal axis of the catheter. The longitudinal reinforcement can include a plurality of longitudinal reinforcements arranged biased to one side of the catheter. The longitudinal reinforcement can include a plurality of longitudinal reinforcements arranged randomly around a central longitudinal axis of the catheter. The longitudinal reinforcement can include a plurality of longitudinal reinforcements the extend along an entire length of the catheter. The longitudinal reinforcement can include a plurality of longitudinal reinforcements that extend only along a partial length of the catheter. The longitudinal reinforcement can include a plurality of longitudinal reinforcements each of equal length. The longitudinal reinforcement can include a plurality of longitudinal reinforcements and at least two of the plurality of longitudinal reinforcements have different relative lengths. The longitudinal reinforcement can be interwoven with the circumferential reinforcement. The longitudinal reinforcement may not be interwoven with the circumferential reinforcement. The longitudinal reinforcement can be placed over, under, or between the circumferential reinforcements or between the inner and outer layer. The longitudinal reinforcement can be embedded in the tie layer. The longitudinal reinforcement can be placed over or under the tie layer. The longitudinal reinforcement can be bonded, welded, or otherwise attached to the circumferential reinforcement. The coil can be provided over the braided pattern. The coil can be provided under the braided pattern. The coil wires can include a first coil reinforcement and a second coil reinforcement placed over the first coil reinforcement. The second coil reinforcement can be wrapped in a direction opposite to a direction in which the first coil reinforcement is wrapped. The braided design can include a first braid reinforcement and a second braid reinforcement placed over the first braid reinforcement. The second braid reinforcement can comprise a material that is different from a material that comprises the first braid reinforcement. The second braid reinforcement can comprises a geometry that is different from a geometry that comprises the first braid reinforcement. The second braid reinforcement can comprise a reinforcement pattern that is different from a reinforcement pattern that comprises the first braid reinforcement.

The reinforcement can be embedded in the tie layer. The tie layer can be placed over and under the reinforcement. The tie layer can be placed between braided and coil reinforcements. The tie layer can be placed between any reinforcement layers.

The reinforcement can terminate a fixed distance from a distal end point of the catheter. The outer layer can comprise a first section and a second section, and a stiffness of the first section can be different from a stiffness of the second section. A trackability of the first section can be better than a trackability of the second section. The tie layer can comprise a first tie layer and a second tie layer. One or more of the inner layer, the tie layer, or the outer layer can be cross-linked using e-beam. The inner layer can comprise a first inner layer on a distal portion of the catheter, the first inner layer can comprise the thermoplastic elastomeric material, and a second inner layer disposed on a proximal portion of the catheter proximal to the first inner layer and the second inner layer can comprise PTFE. The inner layer can be disposed only on a distal portion of the catheter. The inner layer can be disposed on a full length of the catheter.

The catheter can further comprise a catheter distal end that can comprise a radiopaque tip. The radiopaque tip can comprise a polymer including an additive of one or more of tungsten, barium sulfate, bismuth subcarbonate, or bismuth oxychloride. The radiopaque tip comprises a split marker band.

Various additional features and advantages of this invention will become apparent to those of ordinary skill in the art upon review of the following detailed description of the illustrative embodiments taken in conjunction with the accompanying drawings.

### BRIEF DESCRIPTION OF THE DRAWINGS

The following detailed description is better understood when read in conjunction with the appended drawings. For the purposes of illustration, examples are shown in the drawings; however, the subject matter is not limited to the specific elements and instrumentalities disclosed. In the drawings:

FIG. 1A shows a view a catheter in accordance with aspects of the invention;

FIG. 1B shows a cross section view of the catheter of FIG. 1A along section line A-A;

FIG. 1C shows a luer hub of the catheter of FIG. 1A;

FIG. 2 shows a magnified cross section view of section B of the catheter of FIG. 1A;

FIG. 3 shows a process for assembling the catheter shown in FIGS. 1A-2;

FIG. 4A shows a view of another catheter in accordance with aspects of the invention;

FIG. 4B shows a cross section view of the catheter of FIG. 4A along section line C-C;

FIG. 5 shows a process for assembling the catheter shown in FIGS. 4A and 4B;

FIG. 6A shows a view of another catheter in accordance with aspects of the invention;

FIG. 6B shows a cross section view of the catheter of FIG. 6A taken along section line D-D;

FIG. 6C shows a magnified view of portion D of FIG. 6B;

FIG. 7 shows a process for assembling the catheter shown in FIGS. 6A-6C; and

FIG. 8 shows a process for assembling a catheter according to aspects of the invention.

### DETAILED DESCRIPTION OF ILLUSTRATIVE EMBODIMENTS

Many existing catheters utilize PTFE catheter liners. One reason for using PTFE as a catheter liner is to provide low friction for delivery of implants or devices and to allow low friction passage of coaxial inner catheters. Another reason for using PTFE as a catheter liner is that PTFE is an intractable material that lends well to thermal lamination of outer materials when the PTFE surface is activated by etching or other means. But PTFE is a relative stiff material with a Shore D hardness range of 40-60, which means that the flexibility of the catheter is limited by the stiffness of the PTFE. Accordingly, there exists a need, for example in Neurovascular applications, for an alternative or supplement to PTFE catheter liners that have both flexible distal ends and low friction on an inner surface to allow for removal of clots and to facilitate co-axial advancement inside the catheter.

The present invention addresses the need for an alternative or supplement to PTFE catheter liners by providing a catheter liner with a flexible and low friction distal end. The catheter can include a distal liner comprising a thermoplastic elastomeric material, such as a polyethylene-based thermoplastic elastomeric material (e.g., Topas). Catheters having liners that include the polyethylene thermoplastic elastomeric material have low friction and increased flexibility as compared to catheters that primarily use PTFE liners. Aspects of the present invention are also directed to catheters that utilize a low durometer polymer (e.g., Orevac) with the polyethylene thermoplastic elastomeric liner. The low durometer polymer can improve the adherence and/or bonding of the polyethylene thermoplastic elastomeric liner with outer materials (e.g., PEBA). Polyethylene thermoplastic elastomeric liners combined with low durometer polymers can achieve a fully laminated catheter composite with lower friction and increased flexibility as compared to PTFE catheter liners. Such liners can improve for example procedural success rate for access to MCA M1 and M2 artery to treat and remove blockages due to ischemic strokes. These and other aspects of the invention are described as follows in reference to the figures, in which like reference numerals can refer to like structures.

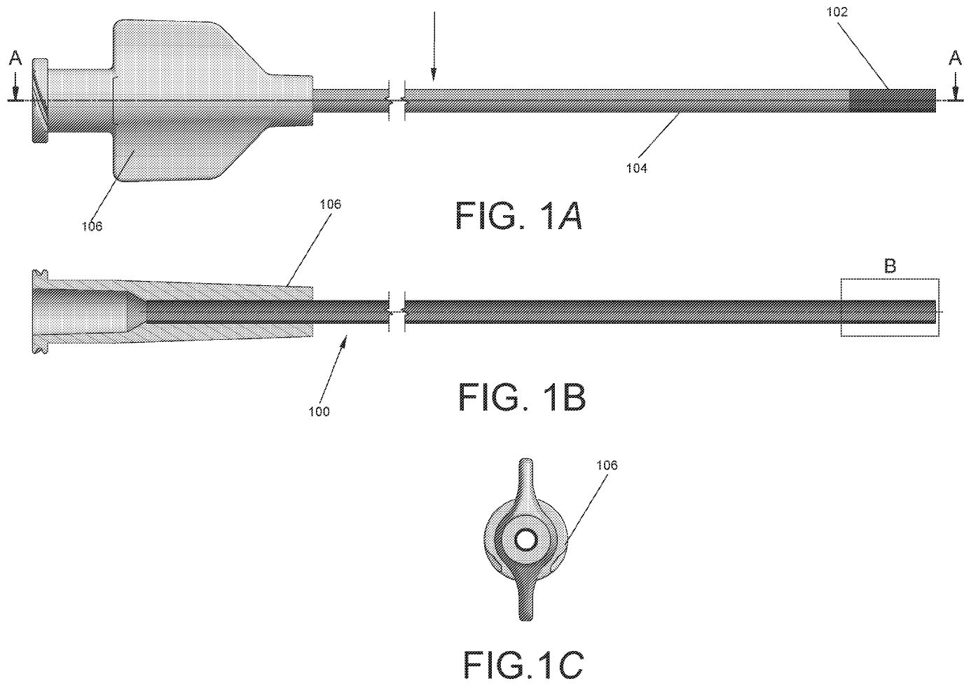

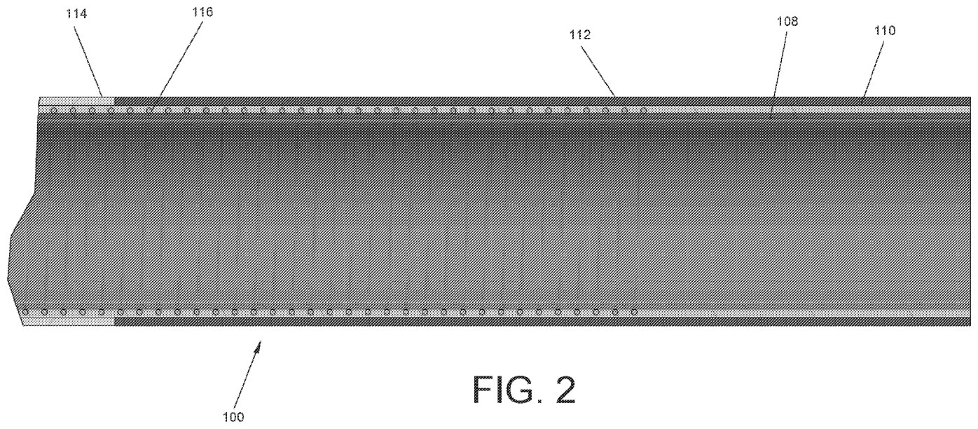

FIGS. 1A-1C shows a catheter 100 in accordance with aspects of the invention. The catheter can include a distal section 102, a proximal section 104, and a luer hub 106. FIG. 2 shows a magnified view of region B of catheter distal section 102 of catheter 100. Distal section 102 can include an inner layer 108, a tie layer 110, a distal outer layer 112, a proximal outer layer 114, and a reinforcement 116, which in embodiments can include a wire, a braid, and/or a coil. Inner layer 108 can comprise a material such as a polyolefin-based thermoplastic elastomer, or a polyethylene-based thermoplastic elastomer of a low durometer. In embodiments, inner layer 108 can comprise lubricants (e.g., Propell™ from Foster Corporation or Mobilize from Compounding Solutions) in the polyolefin-based thermoplastic elastomer. The lubricants can be anchored, i.e., the lubricants will not shed. In embodiments, inner layer 108 can extend the full length of catheter 100. Alternatively, inner layer 108 can extend a partial length of catheter 100, such as for example only in distal section 102 and not in the proximal section 104. Inner layer 108 can be surrounded by tie layer 110, which can promote adhesion between two typically incompatible materials. Tie layer 110 can comprise a low durometer (e.g., Shore D hardness of 60 or less include 56, 30, or lower) polymer. In embodiments, tie layer 110 can include maleic anhydride grafted linear low-density polyethylene (LLDPE), such as Orevac® and/or ReZilok Rx. In embodiments, tie layer 110 can include a maleic anhydride modified low density polyethylene (LDPE). In embodiments, tie layer 110 can include a maleic anhydride modified ethylene vinyl acetate (EVA). In embodiments, tie layer 110 can include other materials as described in for example in EP0873759A2, U.S. Pat. Nos. 6,165,166A, 6,464,683B1, the entirety of which are incorporated herein by reference. Inner layer 108 can include an undulating inner surface profile. Undulating inner surface profile can include a low and a high point. Undulating inner surface profile can reduce contact/friction between inner layer 108 and, for example, an implant or clot. In embodiments, one or more of the inner layer 108, tie layer 110, distal outer layer 112, or proximal outer layer 114 can be cross-linked using e-beam, which can improve adhesion between layers.

In embodiments, distal outer layer 112 and/or proximal outer layer 114 can be polar materials i.e., thermally bondable to other polar surfaces such as polyamide, PEBA (Polyether block amide), various families of polyurethane elastomers, and/or polyester based elastomers (Hytrel®).

In embodiments, reinforcement 116 can be encapsulated within a wall (e.g., tie layer 110) of catheter 100. Reinforcement 116 can comprise a reinforcing framework, which can be a wire, a braid, and/or coil. Reinforcement 116 can contain longitudinal support elements and/or other reinforcing components. In embodiments, reinforcement 116 can be positioned over tie layer 110. In embodiments, reinforcement 116 can be embedded in tie layer 110. In embodiments, the tie layer 110 can be placed over and/or under the reinforcement 116. In embodiments, tie layer 110 can be placed over and/or under reinforcement 116 and can be placed between any reinforcement layers discussed herein. In embodiments, tie layer 110 can be placed over and/or under reinforcement 116 and can be placed between any braid and/or coil reinforcements discussed herein.

In embodiments, reinforcement 116 can be a single coil wire wrapped circumferential to a longitudinal axis of catheter 100. In embodiments, reinforcement 116 can include multiple filar coil wires wrapped circumferential to the longitudinal axis of catheter 100. In embodiments, reinforcement 116 can include a braided design of interwoven wire patterns circumferential to the longitudinal axis of catheter 100. The braided design can include changes in its density or pix per inch all its length. The braided design can include a constant density along its length. The braided design can be a 1 under 1 over 1 or 2 under 2 over 2 or 1 under 2 over 2 or other various interweaving strand patterns. The braided design pattern can be formed with 8, 16, 32 or any number of individual strands. In embodiments, reinforcement 116 can include a geometry that is round, rectangular, or elliptical. In embodiments, reinforcement 116 can include steel, nitinol, tungsten, non-metallic monofilaments, or fibrous bundles like Aramid or from polymers such as Nylon or LCP.

In embodiments, reinforcement 116 can be circumferential between catheter 100 layers and can be augmented with longitudinal reinforcement. The longitudinal reinforcement material can be made steel, nitinol, tungsten, non-metallic monofilaments, or fibrous bundles like Aramid or from polymers such as Nylon or LCP. 1, 2, 3, 4 or any number of longitudinal reinforcements can be provided. The longitudinal reinforcements can be positioned symmetrically around the cross section of catheter 100, or biased to one side, or randomly positioned along the entire length of catheter 100, or for a fixed length, or individual longitudinal reinforcements can have different individual lengths. The longitudinal reinforcement can be interwoven with the circumferential reinforcement. Alternatively, the longitudinal reinforcement may not be interwoven with the circumferential reinforcement. The longitudinal reinforcement can be placed over, under, or between the circumferential reinforcements or between the inner and outer layers 112, 114. The longitudinal reinforcement can be embedded in tie layer 110. The longitudinal reinforcement can be placed over or under tie layer 110. The longitudinal reinforcement can be bonded, welded, or otherwise attached to the circumferential reinforcement.

In embodiments, reinforcement 116 can include any of the coil reinforcements discussed above and any of the braided patterns discussed above and the coil reinforcements can be provided over the braided patterns or vice versa.

In embodiments, reinforcement 116 can include a first coil reinforcement and a second coil reinforcement and the second coil reinforcement can be placed over the first coil reinforcement. The second coil reinforcement can be wrapped in a direction opposite to a direction that the first coil reinforcement is wrapped.

In embodiments, reinforcement 116 in include a first braid reinforcement and a second braid reinforcement, such as any of the previously discussed braided designs. The second braid reinforcement can be placed over the first braid reinforcement. The second braid reinforcement can include a different reinforcement material and/or reinforcement geometry, and/or reinforcement pattern than the first braid reinforcement.

In embodiments, catheter 100 can include a catheter distal end that can include a radiopaque tip. The radiopaque tip can include a polymer, which can include an additive of one or more of tungsten, barium sulfate, bismuth subcarbonate, or bismuth oxychloride. The radiopaque tip can include a split marker band. The split marker band can allow the distal end to circumferentially enlarge when catheter 100 is used as a thrombus removal/suction catheter.

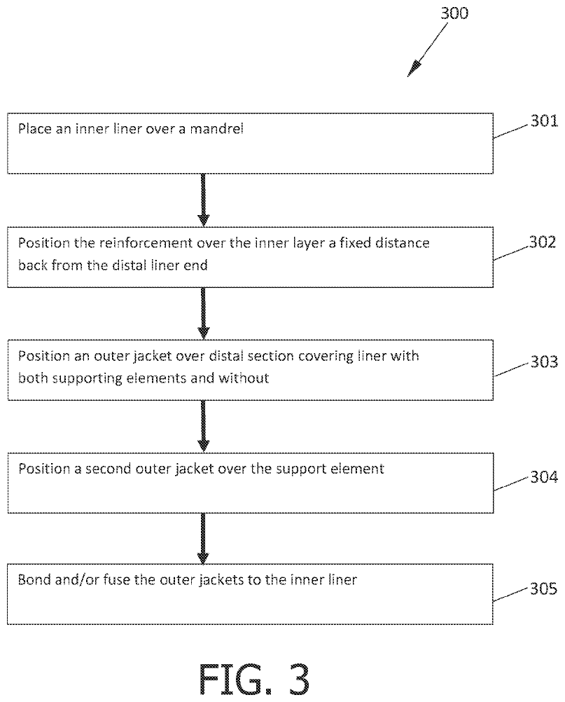

FIG. 3 shows a process 300 of manufacturing a catheter, such as catheter 100. Process 300 can include, at step 301, placing an inner liner over a mandrel. In embodiments, the inner liner can comprise inner layer 108 and tie layer 110, which can be co-extruded with inner layer 108. In embodiments, inner layer 108 can be co-extruded as an AB layered co-extrusion with tie layer 110 as the B or upper layer. In embodiments, inner layer 108 and tie layer 110 can be manually assembled as separate layers and joined in the subsequent fusing step. This approach can allow the stiffness of the catheter shaft to be tailored along its length in line with the specific catheter user needs.

Process 300 can include, at step 302, positioning a reinforcement, such as reinforcement 116, over inner liner a fixed distance back from a distal end of inner layer 108.

Process 300 can include, at step 303, positioning an outer jacket (e.g., distal outer layer 112) over the distal section. Outer jacket can cover the distal section so no reinforcement protrudes through the distal end of catheter 100. In embodiments, catheter 100 can include a material transition to a material with a different durometer prior to the reinforcing element.

Process 300 can include, at step 304, positioning a second outer jacket (e.g., proximal outer layer 114) over the proximal section 104 of catheter 100.

Process 300 can include, at step 305, bonding and/or fusing the assembly together. Fusing can include enclosing the complete assembly inside a heat shrink tubing. The heat shrink tubing can then be passed through a heating chamber at a controlled speed. This process can allow the components to reach a temperature sufficient (with the compressive force applied by the heat shrink) to fuse bond the layers together to form the final composite catheter assembly. Process 300 can conclude by removing the mandrel and/or heat shrink from the final catheter assembly.

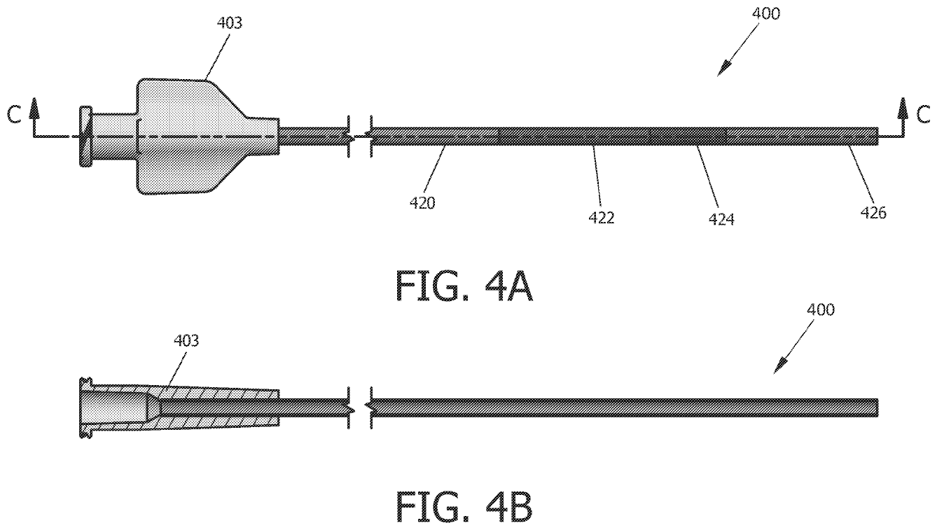

FIGS. 4A and 4B show a catheter 400 with multiple outer layer sections fused over the inner layer and support element assembly. Catheter 400 can include each of the features discussed above in reference to catheter 100, but with a modified outer layer arrangement. For example, catheter 400 can include a first outer layer 420, a second outer layer 422, a third outer layer 424, a fourth outer layer 426, and a luer hub 406. One or more of first outer layer 420, second outer layer 422, a third outer layer 424, and fourth outer layer 426 may comprise a material with a stiffness different than a stiffness of materials that comprises one or more of the other of first outer layer 420, second outer layer 422, a third outer layer 424, and fourth outer layer 426. In embodiments, the stiffness of the materials comprising first outer layer 420, second outer layer 422, a third outer layer 424, and fourth outer layer 426 may increase from the distal to the proximal end such that the distal-most end comprises the most flexible material. One or more of first outer layer 420, second outer layer 422, a third outer layer 424, and fourth outer layer 426 may have a trackability different than a trackability of one or more of the other of first outer layer 420, second outer layer 422, a third outer layer 424, and fourth outer layer 426. For example, the outer layer include a first section and a second section and a trackability of the first section can be better than a trackability of the second section.

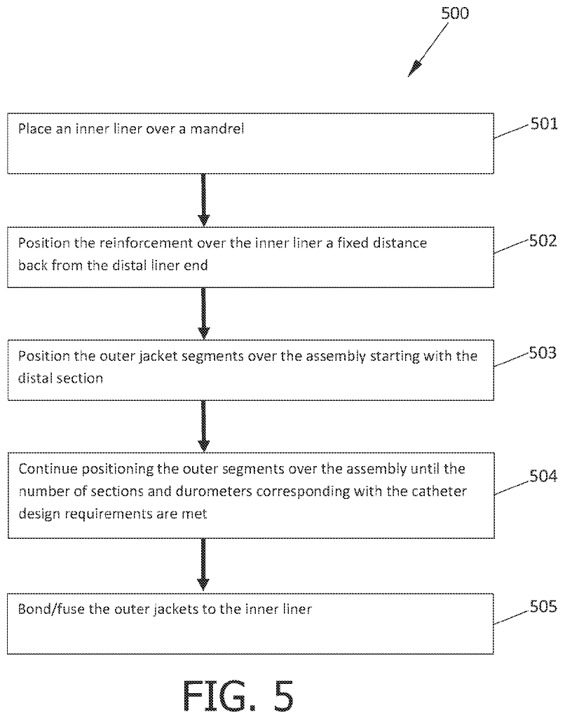

FIG. 5 shows a process 500 of manufacturing a catheter, such as catheter 400. Process 500 can include, at step 501, placing an inner liner over a mandrel. In embodiments, the inner liner can comprise inner layer 108 and tie layer 110, which can be co-extruded with inner layer 108. In embodiments, inner layer 108 can be co-extruded as an AB layered co-extrusion with tie layer 110 as the B or upper layer. In embodiments, inner layer 108 and tie layer 110 can be manually assembled as separate layers and joined in the subsequent fusing step. This approach can allow the stiffness of the catheter shaft to be tailored along its length in line with the specific catheter user needs.

Process 500 can include, at step 502, positioning a reinforcement, such as reinforcement 116, over inner liner a fixed distance back from a distal end of inner layer 108.

Process 500 can include, at step 503, positioning the outer jacket segments (e.g., first outer layer 420, second outer layer 422, third outer layer 424, fourth outer layer 426) over the assembly starting with distal section.

Process 500 can include, at step 504, continuing positioning the outer segments over the assembly until the number of sections and durometers corresponding with the catheter design requirements are met.

Process 500 can include, at step 505, bonding and/or fusing the outer jackets to the inner liner. Fusing can include enclosing the complete assembly inside a heat shrink tubing. The heat shrink tubing can then be passed through a heating chamber at a controlled speed. This process can allow the components to reach a temperature sufficient (with the compressive force applied by the heat shrink) to fuse bond the layers together to form the final composite catheter assembly. Process 500 can conclude by removing the mandrel and/or heat shrink from the final catheter assembly.

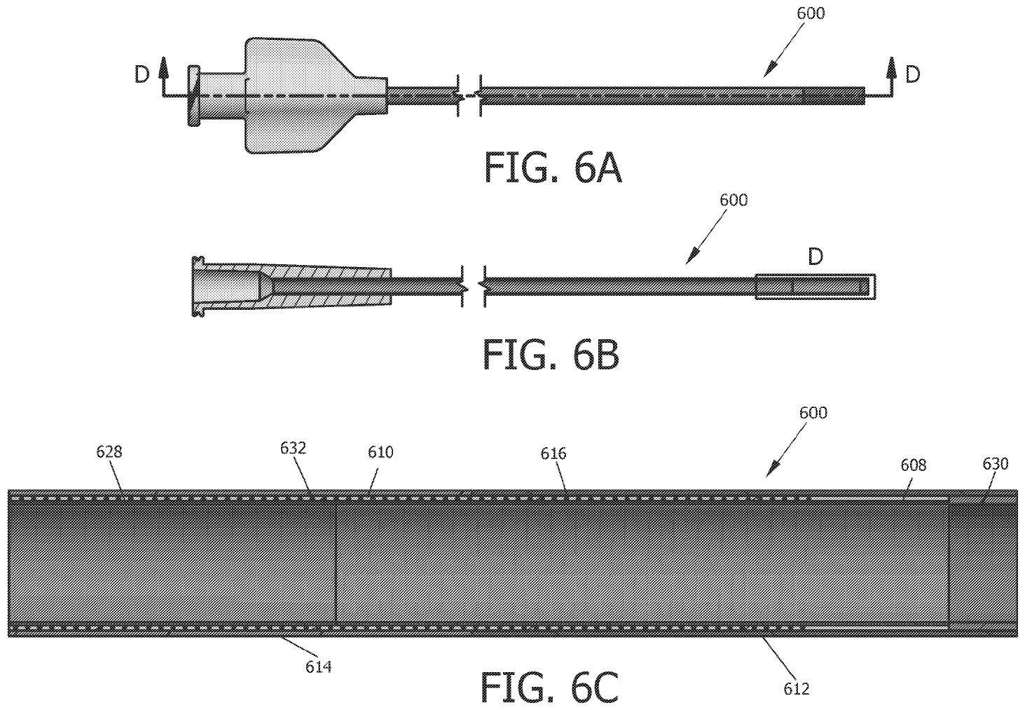

FIGS. 6A-6C shows liner configurations for a catheter 600. Catheter 600 can include features similar to those discussed previously in reference to catheter 100. For example, catheter 600 can include a first inner layer 608, which can be as long as or shorter than the length of the catheter. First inner layer 608 can comprise a material such as a polyolefin-based thermoplastic elastomer, or a polyethylene-based thermoplastic elastomer of a low durometer. Catheter 600 can include a second inner layer 628. Second inner layer 628 can for example be a fluoropolymer liner such as PTFE. Second inner layer 628 can be an etched PTFE liner, which can be manufactured with a solution coating manufacturing process. In embodiments, second inner layer 628 can be a ram or pasted extruded etched PTFE liner and catheter 600 can be assembled in accordance with process 800, discussed below. This approach can avoid the occurrence of flash or bleed through into the inner diameter of catheter 600. Second inner layer 628 can be shorter than the length of the catheter. Catheter 600 having first inner layer 608 and second inner layer 628 can allow the distal end of catheter 600 to exhibit different properties to that of the proximal end. This can result in a more flexible or trackable tip distal section while the proximal end of catheter 600 can be optimized for pushability. A transition between first inner layer 608 and second inner layer 628 can be smooth with no obstruction.

Catheter 600 can include a third inner layer 630. Catheter 600 can include a distal outer layer 612. Distal outer layer 612 can be a single layer or multiple layers. Third inner layer 630 and distal outer layer 612 can comprise the same material. Catheter 600 can include a proximal outer layer 614. Proximal outer layer 614 can be a single layer or multiple layers. Catheter 600 can include a reinforcement layer 616, as discussed above. Catheter 600 can include a first tie layer 610 for first inner layer 608. Catheter 600 can include a second tie layer or etch 632 for second inner layer 628. First and second tie layers 610, 632 can comprise a low durometer polymer, as discussed above.

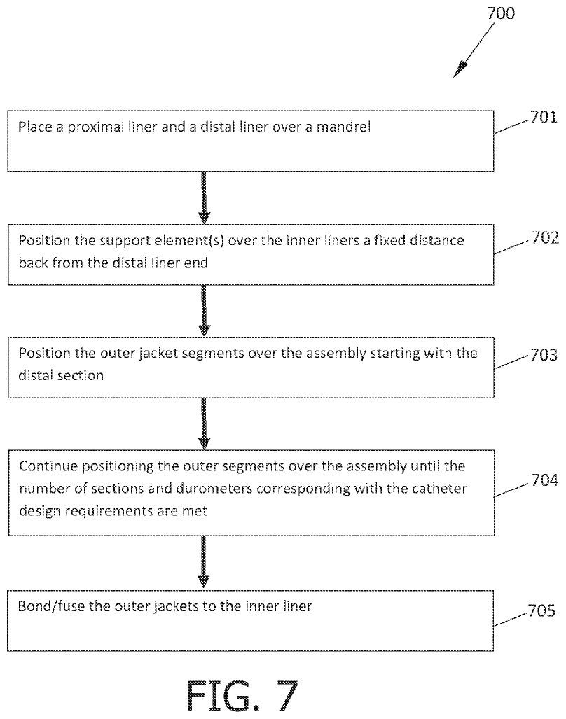

FIG. 7 shows a process 700 of manufacturing a catheter, such as catheter 600. Process 700 includes, at step 701, placing a proximal liner (e.g., second inner layer 628) and a distal liner (e.g., first inner layer 608) over a mandrel. In embodiments, the inner liner can comprise inner layer 108 and tie layer 110, which can be co-extruded with inner layer 108. In embodiments, inner layers can be co-extruded with tie layers (e.g., first tie layer 610 and second tie layer 632). The distal liner can be square cut and butted up against the end of the proximal liner.

Process 700 can include, at step 702, positioning support elements (e.g., reinforcement 616) over the proximal liner and distal liner a fixed distance back from the distal liner end. In embodiments, process 700 can include terminating and enclosing the ends of the supporting elements in the catheter wall back from the very distal catheter tip to ensure the catheter is atraumatic.

Process 700 can include, at step 703, positioning outer jacket segments (e.g., distal outer layer 612 and/or proximal outer layer 614) over the proximal liner, distal liner, and support element assembly starting with the distal section.

Process 700 can include, at step 704, continuing to position the outer jacket segments over the proximal liner, distal liner, and support element assembly until a sufficient number of sections and durometers are formed.

Process 700 can include, at step 705, bonding and/or fusing the outer jackets to the proximal and distal inner liners. Fusing can include enclosing the complete assembly inside a heat shrink tubing. The heat shrink tubing can then be passed through a heating chamber at a controlled speed. This process can allow the components to reach a temperature sufficient (with the compressive force applied by the heat shrink) to fuse bond the layers together to form the final composite catheter assembly. Process 700 can conclude by removing the mandrel and/or heat shrink from the final catheter assembly.

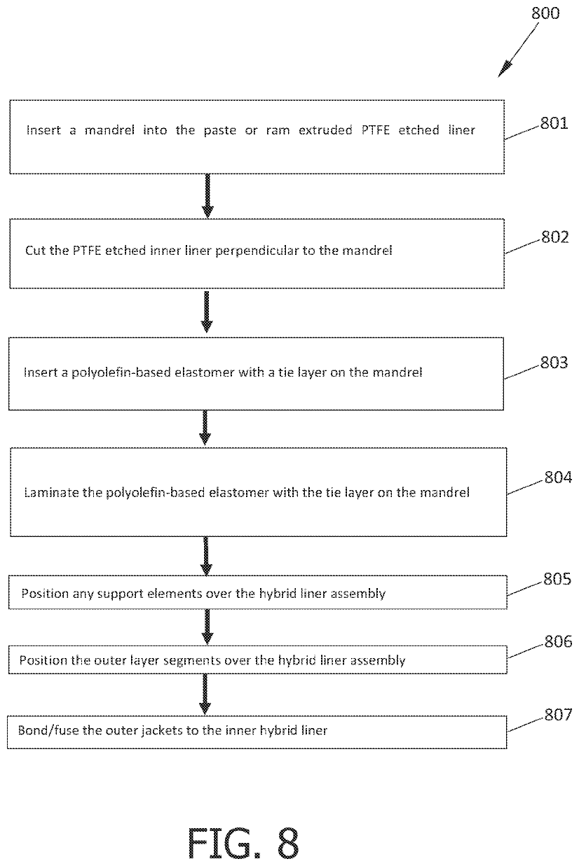

FIG. 8 shows a process 800 of manufacturing a catheter. Process 800 can include, at step 801, inserting a mandrel into a paste or ram extruded PTFE etched inner liner. In embodiments, the PTFE etched inner liner can have an inner diameter smaller than an outer diameter of the mandrel. For example, the PTFE etched inner liner can have an inner diameter between 2-8% smaller than an outer diameter of the mandrel. In embodiments, the mandrel can have a conical end that can be between 10-20 mm and that can aid insertion of the mandrel into the PTFE etched inner liner.

Process 800 can include, at step 802, cutting the PTFE etched inner liner perpendicular to the mandrel. The PTFE etched inner liner can be cut to a specified length. In embodiments, since the PTFE etched inner liner can have an inner diameter smaller than an outer diameter of the mandrel the PTFE etched inner liner can remain tight on the mandrel while being cut which can prevent the liner from springing back after the cutting as occurs with traditional liner loading processes.

Process 800 can include, at step 803, inserting a polyolefin-based elastomer with a tie layer on the mandrel. The polyolefin-based elastomer with the tie layer can be inserted where the PTFE etched inner liner was removed at step 802. The polyolefin-based elastomer with the tie layer can abut against the remaining PTFE etched inner liner on the mandrel, which can optimize the junction between the two materials.

Process 800 can include, at step 804, laminating the polyolefin-based elastomer with the tie layer. Laminating the polyolefin-based elastomer with the tie layer can achieve a tight fit to the mandrel and/or to a terminal edge of the PTFE etched inner liner, which can create an internal seal that can limit and/or prevent penetration of other materials into an inner diameter of the finished catheter.

Process 800 can include, at step 805, positioning any support elements over the hybrid liner assembly. In embodiments, step 805 can include any of the aspects of step 702 discussed above.

Process 800 can include, at step 806, positioning outer layer segments over the hybrid liner assembly. In embodiments, step 806 can include any of the aspects of steps 703 and/or 704 discussed above.

Process 800 can include, at step 807, bond and/or fusing the outer jackets to the hybrid liner assembly. In embodiments, step 807 can include any of the aspects of step 705 discussed above.

It will be appreciated that the foregoing description provides examples of the disclosed machine. However, it is contemplated that other implementations of the invention may differ in detail from the foregoing examples. All references to the invention or examples thereof are intended to reference the particular example being discussed at that point and are not intended to imply any limitation as to the scope of the invention more generally. All language of distinction and disparagement with respect to certain features is intended to indicate a lack of preference for those features, but not to exclude such from the scope of the invention entirely unless otherwise indicated. All methods described herein can be performed in any suitable order unless otherwise indicated herein or otherwise clearly contradicted by context.

1. A catheter comprising:
an inner layer comprising a polyolefin- or polyethylene-based thermoplastic elastomeric material;
an outer layer; and
a tie layer disposed between the inner layer and the outer layer, wherein the tie layer comprises a low durometer polymer.

2. The catheter of claim 1, wherein the inner layer comprises one or more lubricants.

3. The catheter of claim 1, wherein the tie layer comprises a maleic anhydride grafted linear low-density polyethylene (LLDPE), or a maleic anhydride modified low density polyethylene (LDPE), or a maleic anhydride modified ethylene vinyl acetate (EVA).

4. (canceled)

5. (canceled)

6. The catheter as in claim 1, further comprising a reinforcement disposed between the inner layer and the outer layer.

7. (canceled)

8. The catheter of claim 6, wherein the reinforcement includes one or more filar coil wires wrapped circumferentially about a longitudinal axis of the catheter.

9. The catheter of claim 6, wherein the reinforcement is a braided design wire pattern interwoven circumferentially around a longitudinal axis of the catheter.

10. The catheter of claim 9, wherein the braided design includes changes in density or pix per inch along a length of the braided design.

11. (canceled)

12. The catheter of claim 9, wherein the braided design includes a pattern of 1 under 1 over 1, 2 under 2 over, 2 or 1 under 2 over 2.

13. (canceled)

14. (canceled)

15. The catheter as in claim 6, wherein the reinforcement comprises at least one of steel, nitinol, tungsten, non-metallic monofilaments, fibrous bundles, Aramid, polymers, Nylon, or LCP.

16. The catheter as in claim 6, wherein the reinforcement includes a circumferential reinforcement between the inner layer and the outer layer and a longitudinal reinforcement that extends along a longitudinal axis of the catheter.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. The catheter as in claim 9, wherein the braided design includes a first braid reinforcement and a second braid reinforcement placed over the first braid reinforcement.

37. The catheter of claim 36, wherein the second braid reinforcement comprises a material that is different from a material that comprises the first braid reinforcement.

38. The catheter of claim 36, wherein the second braid reinforcement comprises a geometry that is different from a geometry that comprises the first braid reinforcement.

39. (canceled)

40. The catheter as in claim 6, wherein the reinforcement is embedded in the tie layer.

41. The catheter as in claim 6, wherein the tie layer is placed over and under the reinforcement.

42. The catheter of claim 41, wherein the tie layer is placed between braided and coil reinforcements.

43. The catheter of claim 41, wherein the tie layer is placed between any reinforcement layers.

44. The catheter of claim 6, wherein the reinforcement terminates a fixed distance from a distal end point of the catheter.

45. The catheter as in claim 1, wherein the outer layer comprises a first section and a second section, wherein a stiffness of the first section is different from a stiffness of the second section.

46. The catheter as in claim 1, wherein the outer layer comprises a first section and a second section, wherein a trackability of the first section is better than a trackability of the second section.

47. (canceled)

48. The catheter as in claim 1, wherein one or more of the inner layer, the tie layer, or the outer layer is cross-linked using e-beam.

49. The catheter as in claim 1, wherein the inner layer comprises a first inner layer on a distal portion of the catheter, the first inner layer comprising the thermoplastic elastomeric material, and a second inner layer disposed on a proximal portion of the catheter proximal to the first inner layer, wherein the second inner layer comprises PTFE.

50. (canceled)

51. (canceled)

52. The catheter as in claim 1, wherein a catheter distal end comprises a radiopaque tip.

53. The catheter of claim 52, wherein the radiopaque tip comprises a polymer including an additive of one or more of tungsten, barium sulfate, bismuth subcarbonate, or bismuth oxychloride.

54. The catheter of claim 53, wherein the radiopaque tip comprises a split marker band.
